(19) United States
(12) Patent Application Publication
(10) Pub. No.: US 2008/0154113 A1
(43) Pub. Date: Jun. 26, 2008

(54) Apparatus and method for positioning electrodes on the body

(76) Inventor: Yitzhak Zilberman, Santa Clarita, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/761,787

(22) Filed: Jun. 12, 2007

Related U.S. Application Data
(60) Provisional application No. 60/813,257, filed on Jun. 12, 2006.

(51) Int. Cl. A61B 5/04

(52) U.S. Cl. 600/390; 600/386; 600/388

(57) ABSTRACT

An apparatus includes a fitting device and an actuator movably coupled to the fitting device. The fitting device is configured to be disposed about a portion of a body. An inner surface of the fitting device has an attachment portion configured to removably couple an electrode to the inner surface of the fitting device. The actuator is configured to decouple the electrode from the attachment portion of the fitting device when the fitting device is disposed about the portion of the body.

### CROSS-REFERENCE TO RELATED APPLICATIONS

This application claims priority to U.S. Provisional Application Ser. No. 60/813,257, entitled “Apparatus and Method for Positioning Electrodes on the Body,” filed Jun. 12, 2006, which is incorporated herein by reference in its entirety.

### BACKGROUND

The invention relates generally to medical devices and procedures, and more particularly to apparatuses and methods for positioning electrodes on the body.

Surface electrodes can be used in a variety of medical procedures, such as, for example, electromyography (EMG) and neuromuscular electrical stimulation (NMES) therapy. For example, some known therapeutic devices include one or more surface electrodes that provide electrical stimulation to portions of a patient's leg to improve the patient's gait mechanics. Such “gait training” therapy can be used, for example, to cause dorsiflexion of the ankle in patients who have lost the ability to dorsiflex following neurological injury.

Known electrodes must be accurately and repeatably positioned on the patient's body to ensure that the electrical signals are delivered to and/or received from the appropriate portions of the body. For example, some known gait training therapeutic devices include a first electrode to be positioned on the patient's leg above the deep peroneal nerve and a second electrode to be positioned on the patient's leg above the superficial peroneal nerve. In some instances, failure to accurately position the electrodes can result in an exaggerated inversion or eversion of the foot.

Some known therapeutic devices include electrodes that can be surgically implanted to maintain their position throughout the patient's rehabilitation. When using such devices, however, the patient is potentially subjected to the risks associated with the surgical procedure to implant the electrodes. Moreover, such electrodes cannot be easily removed between rehabilitation procedures.

Some known therapeutic devices include electrodes that are positioned on an orthosis and/or a garment. During therapy, the patient wears the orthosis or garment to ensure that the electrodes are maintained in their desired position. The positioning of electrodes using an orthosis and/or garment, however can result in a bulky device being attached to the area of the patient's body undergoing therapy. The use of an orthosis and/or garment during therapy may apply pressure to the skin and may prevent ventilation of parts of the skin, potentially also causing skin irritation. Moreover, in some circumstances the electrodes may not be accurately positioned (i.e., when the garment stretches, thereby distorting the relative positions of the electrodes).

Some known therapeutic devices include electrodes that are individually attached on the patient's body by skin adhesion. The placement of such electrodes can be guided by landmarks on the skin. Accordingly, the individual placement of electrodes can be inaccurate and/or not repeatable between therapeutic sessions.

Thus, a need exists for improved apparatuses and methods for placing electrodes on the body.

### SUMMARY

Apparatuses and methods for placing electrodes on the body are described herein. In some embodiments, an apparatus includes a fitting device and an actuator movably coupled to the fitting device. The fitting device is configured to be disposed about a portion of a body. An inner surface of the fitting device has an attachment portion configured to removably couple an electrode to the inner surface of the fitting device. The actuator is configured to decouple the electrode from the attachment portion of the fitting device when the fitting device is disposed about the portion of the body.

### BRIEF DESCRIPTION OF THE DRAWINGS

FIGS. 1 and 2 are schematic illustrations of a medical device according to an embodiment of the invention in a first configuration and a second configuration, respectively.

FIG. 3 is a perspective front view of a medical device according to an embodiment of the invention.

FIGS. 4 and 5 are partial cross-sectional views of the medical device shown in FIG. 3 taken along line X-X in FIG. 3, in a first configuration and a second configuration, respectively.

FIGS. 6-8 are perspective views showing the medical device shown in FIGS. 3-5 as used to place electrodes on a patient's leg.

FIG. 9 is a perspective rear view of a medical device according to an embodiment of the invention.

FIGS. 10-13 are partial cross-sectional views of the medical device shown in FIG. 9 taken along line X-X in FIG. 9, in a first configuration, a second configuration, a third configuration and a fourth configuration, respectively.

FIGS. 14-15 are cross-sectional side views of a medical device according to an embodiment of the invention in a first configuration and a second configuration, respectively.

FIG. 16 is a perspective side view of a medical device according to an embodiment of the invention disposed on a patient's leg.

FIG. 17 is a cross-sectional view of the medical device shown in FIG. 16 taken along line X-X in FIG. 16.

FIG. 18 is a perspective view of a portion of the medical device shown in FIGS. 16 and 17 disposed about a portion of the patient's leg.

FIG. 19 is a flow chart of a method according to an embodiment of the invention.

### DETAILED DESCRIPTION

In some embodiments, an apparatus includes a fitting device and an actuator movably coupled to the fitting device. The fitting device, which can include a flexible garment portion, is configured to be disposed about a portion of a body. An inner surface of the fitting device has an attachment portion configured to removably couple an electrode to the inner surface of the fitting device. The attachment portion can include, for example, an adhesive, a hook and loop fastener, a snap fastener and/or a magnetic fastener. The actuator is configured to decouple the electrode from the attachment portion of the fitting device when the fitting device is disposed about the portion of the body.

In some embodiments, an apparatus includes a fitting device configured to be disposed about a first portion of a body, such as, for example, the leg. The fitting device has an indexing portion and an electrode attachment portion. The indexing portion is configured to receive a second portion of the body, such as, for example, the kneecap, to position the fitting device about the first portion of the body at a predetermined location. The electrode attachment portion is configured to removably couple an electrode to an inner surface of the fitting device such that the electrode is disposed at a predetermined location relative to the second portion of the body when the fitting device is disposed about the first portion of the body.

In some embodiments, an apparatus includes a fitting device and an actuator coupled to the fitting device. The fitting device is configured to be disposed about a portion of a body. A distal end portion of the actuator includes a connector configured to removably couple an electrode to the distal end portion of the actuator. The actuator is configured to move the electrode into contact with the portion of the body when the fitting device is disposed about the portion of the body.

In some embodiments, an apparatus includes a sleeve, an electrode and a fitting device. The sleeve has an inner surface and an outer surface, and is configured to be disposed about a first portion of a body. The electrode is coupled to the inner surface of the sleeve. The fitting device has an indexing portion and an attachment portion. The indexing portion of the fitting device is configured to be disposed about a second portion of the body. The attachment portion of the fitting device is configured to removably retain the sleeve within an interior portion of the fitting device such that the electrode is disposed at a predetermined location relative to the second portion of the body when the fitting device is disposed about the first portion of the body.

In some embodiments, a method includes disposing a fitting device about a portion of a body. The fitting device includes an electrode coupled to an attachment portion of an inner surface of the fitting device. An actuator is moved relative to the fitting device to decouple the electrode from the attachment portion after the disposing. The fitting device is removed from the portion of the body.

As used in this specification, the words “proximal” and “distal” refer to the direction closer to and away from, respectively, an operator (e.g., surgeon, physician, nurse, technician, etc.) who would use a medical device or a therapeutic device during a procedure. For example, the end of a medical device first to contact the patient's body would be the distal end, while the opposite end of the medical device (e.g., the end of the medical device being operated by the operator) would be the proximal end of the medical device.

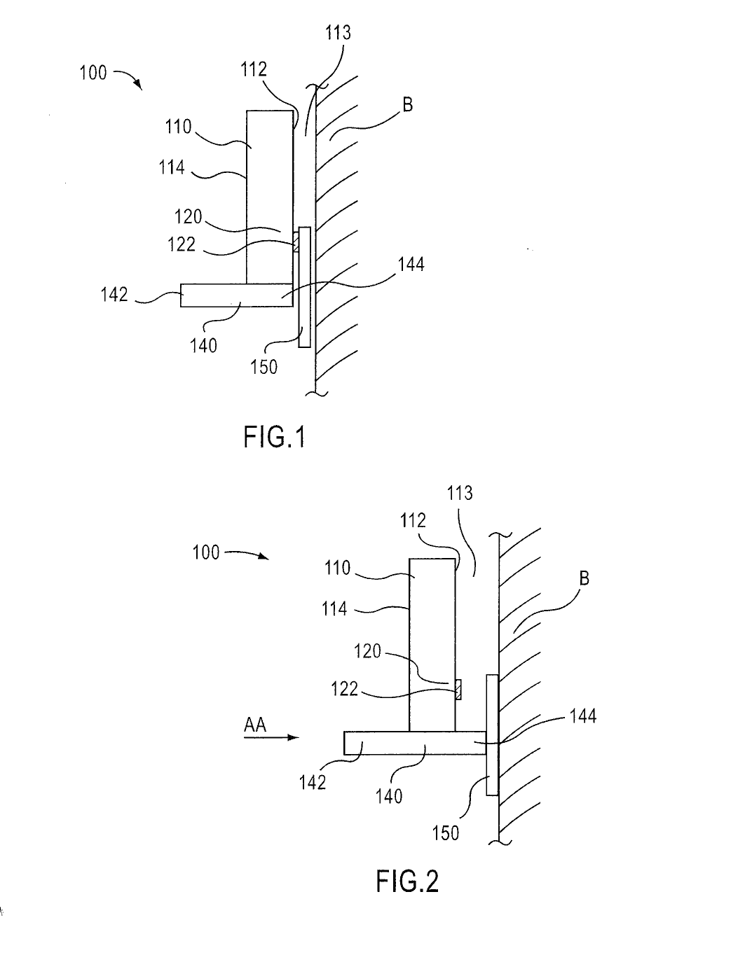

FIGS. 1 and 2 are schematic illustrations of a medical device 100 according to an embodiment of the invention in a first configuration and a second configuration, respectively. The medical device 100 includes a fitting device 110, an actuator 140 and an electrode 150. The fitting device 110 has an inner surface 112 and an outer surface 114. The inner surface 112 of the fitting device 110 defines an interior region 113 within which a portion of a body B is disposed. In this manner, the fitting device 110 can be disposed about the portion of the body B such that the inner surface 112 is in contact with and/or is adjacent the body B.

The inner surface 112 of the fitting device 110 includes an attachment portion 120 having a coupler 122. The coupler 122 removably couples the electrode 150 to the fitting device 110. The coupler 122 can be, for example, a layer of adhesive, a hook and loop fastener, a snap fastener, a magnetic fastener or any other mechanism suitable for removably coupling the electrode 150 to the fitting device 110. In some embodiments, the electrode 150 can be subdivided into multiple segments, with each segment configured to stimulated separately.

The actuator 140 has a proximal end portion 142 and a distal end portion 144 and is movably coupled to the fitting device 110. The proximal end portion 142 of the actuator 140 is disposed outside of the interior region 113 defined by the inner surface 112. Said another way, the proximal end portion 142 of the actuator 140 is accessible to a user when the fitting device 110 is disposed about the portion of the body B. Accordingly, a user can manipulate the proximal end portion 142 of the actuator 140 to move the actuator 140 relative to the fitting device 110 when the fitting device 110 is disposed about the portion of the body B. In this manner, the actuator 140 can be moved as shown by the arrow AA in FIG. 2 to move the medical device 100 from a first configuration (FIG. 1) to a second configuration (FIG. 2).

When the medical device 100 is in the first configuration, the distal end portion 144 of the actuator 140 is disposed adjacent the inner surface 112 of the fitting device 110. Additionally, when the medical device 100 is in the first configuration, the distal end portion 144 of the actuator 140 is disposed apart from the electrode 150. When the medical device 100 is in the second configuration, the distal end portion 144 of the actuator 140 is disposed within the interior region 113 defined by the inner surface 112 and is in contact with the electrode 150. In this manner, when the actuator 140 moves from its first position (corresponding to the first configuration of the medical device 100) to its second position (corresponding to the second configuration of the medical device 100) as illustrated by the arrow AA in FIG. 2, the distal end portion 144 of the actuator 140 decouples the electrode 150 from the coupler 122. Moreover, when the actuator 140 moves from its first position to its second position, the distal end portion 144 of the actuator 140 moves the electrode 150 into contact with the portion of the body B. In this manner, the fitting device 110 can position the electrode 150 in a desired location on the portion of the body B. In some embodiments, the electrode 150 can include a conductive hydrogel (not shown in FIGS. 1 and 2) to couple the electrode to the body B. Such hydrogels can include, for example, a wet gel disposed on a gel sponge coupled to the electrode, a solid gel applied directly to the electrode, or any other suitable conductive hydrogel.

As described in more detail herein, when the electrode 150 is positioned on the portion of the body B, the fitting device 110 can be removed from the portion of the body B, leaving the electrode 150 in position on the body B. Alternatively, in other embodiments, the fitting device 110 can remain disposed about the portion of the body B when the electrode 150 is positioned on the body B as desired. For example, in some embodiments, the fitting device 110 can also provide structural support for the portion of the body B during therapy.

The fitting device 110 can be constructed from any suitable material or combination of materials. For example, in some embodiments, the fitting device 110 can be a constructed from a plastic, such as Nylon. In other embodiments, the fitting device 110 can be constructed from a composite material, such as fiberglass. In yet other embodiments, the fitting device 110 can be constructed from several different materials, such as, for example, rigid plastic, metallic materials, foam padding, cloth, elastic materials or the like. In some embodiments, for example, the fitting device 110 can be constructed from a rigid material such that at least a portion of the attachment portion 120 is rigid. In this manner, the attachment portion 120 can maintain the electrode 150 in a predetermined location within the fitting device 110. Said another way, in such embodiments, the attachment portion 120 can maintain a location, orientation and/or position of the electrode 150 relative to the portion of the body B when the fitting device 110 is disposed about the portion of the body B. In other embodiments, at least a portion of the fitting device 110 can include a flexible and/or elastic garment such that the fitting device can conform to different portions of the body B and/or different sizes and/or shapes of different patients.

The actuator 140 can be any suitable actuator constructed from any suitable material. For example, in some embodiments, the proximal end portion 142 of the actuator 140 can have a size and/or a shape that can be easily manipulated by a user. Similarly, in some embodiments, the distal end portion 144 of the actuator 140 can have a size and/or a shape that corresponds to a size and/or a shape of the electrode 150. For example, in some embodiments, the distal end portion 144 of the actuator 140 can have a shape that corresponds to a shape of the electrode 150. Accordingly, when the actuator 140 is moved into contact with the electrode 150, the distal end portion 144 of the actuator 140 can transmit a spatially uniform force to the electrode 150. In this manner, the electrode can be evenly and securely positioned on the portion of the body B.

The electrode 150 can be any suitable electrode for transmitting an electrical signal to the body B and/or receiving an electrical signal from the body B. For example, in some embodiments, the electrode 150 can be a surface electrode capable of transmitting an electrical signal having an amplitude of between 0.2 mA and 80 mA at a frequency of between 20 and 60 pulses per second. In other embodiments, the electrode 150 can be a surface electrode capable of receiving an electrical signal of between 20 μA and 10 mA at a frequency of between 10 Hz and 5 KHz. In yet other embodiments, the electrode 150 can be capable of transmitting a polarized electrical signal (i.e., the electrode 150 can have a negative or positive polarity).

Similarly, the electrode 150 can be constructed from any suitable components and can have any suitable features. In some embodiments, for example, the electrode 150 can include a cloth backing (not shown in FIGS. 1 and 2) surrounding the electronic portion of the electrode. In other embodiments, the electrode 150 can include a snap connector to provide an electrical connection to a lead wire. In other embodiments, the electrode 150 can include an integrated electrical lead. In yet other embodiments, the electrode 150 can include one or more layers of conductive hydrogel of the types described above.

Similarly, the electrode 150 can have any suitable size and/or shape. For example, in some embodiments, the electrode 150 can be a surface electrode having a circular shape ranging in size between 0.5 inches and 2 inches. In other embodiments, the electrode 150 can be a surface electrode having a rectangular shape. Such rectangular shaped electrodes can range in size from a 1.5 inch square electrode to a 3 inch by 5 inch rectangular electrode.

In some embodiments the electrode may be divided into plurality of segments, electrically insulated from each other and serving as separate electrodes.

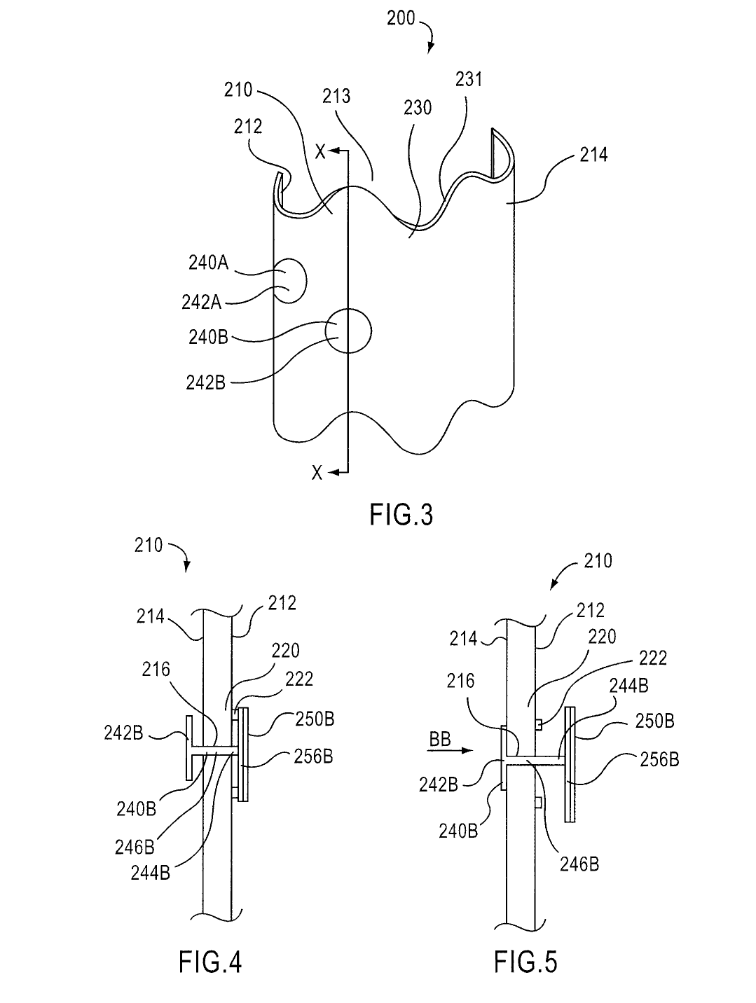

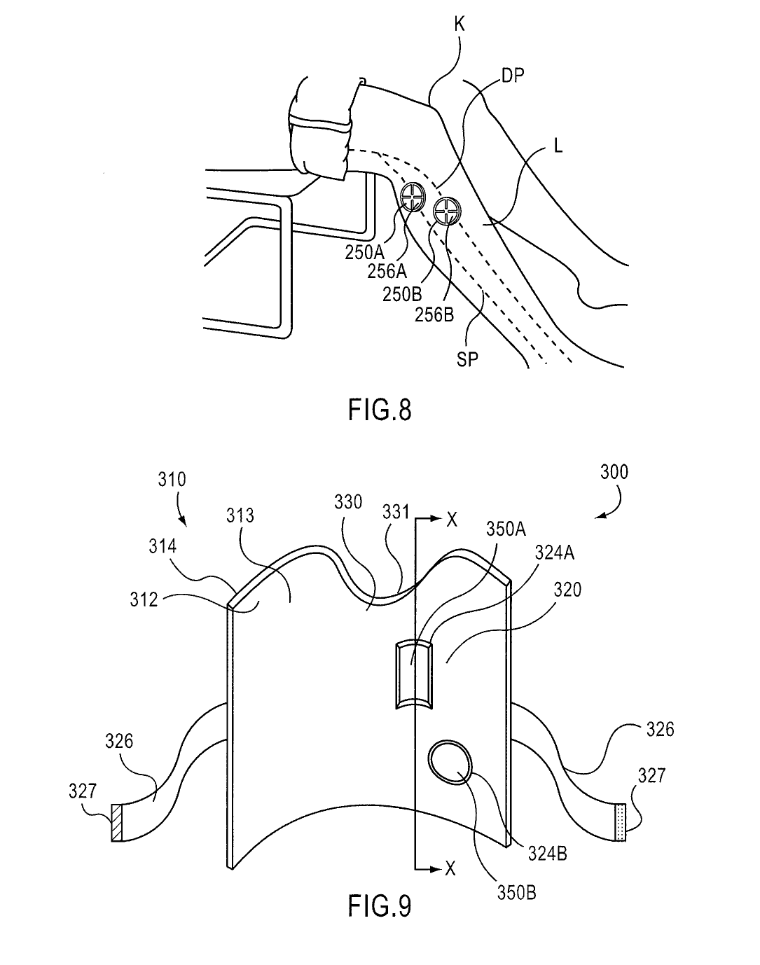

In some embodiments, a fitting device can include an indexing portion to orient and/or position the fitting device about the body at a predetermined location. For example, FIGS. 3-8 illustrate a medical device 200 according to an embodiment of the invention. FIG. 3 is a perspective front view of the medical device 200. FIGS. 4 and 5 are partial cross-sectional views of the medical device 200 taken along line X-X in FIG. 3 in a first configuration and a second configuration, respectively. The medical device 200 includes a fitting device 210, two actuators 240A, 240B and two electrodes 250A, 250B (see, e.g., FIG. 8). The fitting device 210 has an inner surface 212 and an outer surface 214. The inner surface 212 of the fitting device 210 defines an interior region 213 within which a portion of a patient's leg L can be disposed (see, e.g., FIG. 7). In some embodiments, the inner surface 212 of the fitting device 210 can have a shape that corresponds to a shape of the patient's leg L. Said another way, in some embodiments, the inner surface 212 of the fitting device can define a radius of curvature that corresponds to a radius of curvature of a portion of the patient's leg L.

The inner surface 212 of the fitting device 210 includes an attachment portion 220 having two couplers 222. The couplers 222 removably couple the electrodes 250A, 250B to the fitting device 210. The couplers 222 can be, for example, a layer of adhesive, a hook and loop fastener, a snap fastener, a magnetic fastener or any other mechanism suitable for removably coupling the electrodes 250A, 250B to the fitting device 210. In some embodiments, for example the couplers 222 can be bands of Velcro® configured to engage a backing 256B (see FIGS. 4 and 5) of the electrode 250B to removably couple the electrode 250B to the fitting device 210.

The fitting device 210 includes an indexing portion 230 that defines a curved edge 231. As described in more detail herein, the curved edge 231 has a shape that corresponds to a shape of the patient's kneecap K (see, e.g., FIG. 7). Accordingly, the indexing portion 230 can receive and/or contact a portion of the patient's kneecap K to position the fitting device 210 about the patient's leg L at a predetermined location, orientation and/or position. In this manner, when the fitting device 210 is disposed about the patient's leg L with the patient's kneecap K disposed within or adjacent the curved edge 231, the electrodes 250A, 250B are positioned at a predetermined location, orientation and/or position on the patient's leg L.

As shown in FIGS. 4 and 5, the actuator 240B has a proximal end portion 242B, a central portion 246B, and a distal end portion 244B. The central portion 246B of the actuator 240B is movably disposed within a lumen 216 defined by the fitting device 210. The proximal end portion 242B of the actuator 240B is disposed outside of the interior region 213 defined by the inner surface 212. Said another way, the proximal end portion 242B of the actuator 240B is accessible to a user when the fitting device 210 is disposed about the portion of the patient's leg L (see, e.g. FIG. 7). Accordingly, as described in more detail herein, a user can manipulate the proximal end portion 242B of the actuator 240B to move the actuator 240B within the lumen 216 relative to the fitting device 210 when the fitting device 210 is disposed about the patient's leg L.

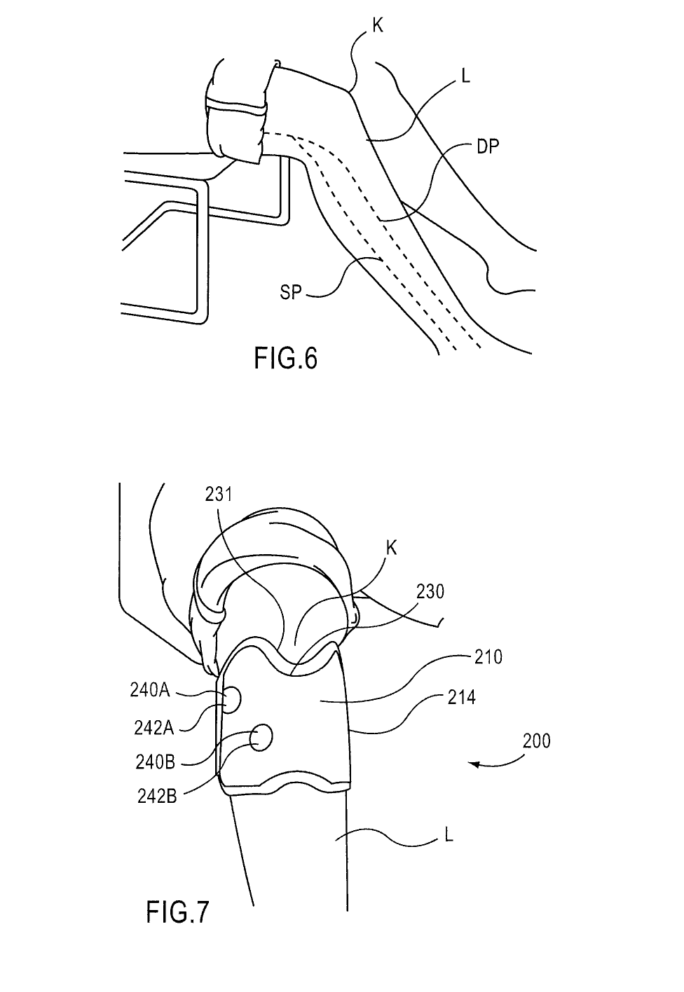

The application of the medical device 200 on a patient's leg L is described with reference to FIGS. 6-8. As shown in FIG. 6, the patient's leg L is prepared for the application of the electrodes 250A and 250B by exposing a portion of the leg L. The fitting device 210 is disposed about a portion of the leg L such that the kneecap K is disposed within or adjacent the curved edge 231 of the indexing portion 230 of the fitting device 210. In some embodiments, the fitting device 210 can be positioned about the leg L in a continuous motion. In other embodiments, the fitting device 210 can be positioned about the leg L in multiple discrete steps. For example, in some embodiments, the fitting device 210 can be positioned at an angle relative to the leg L (the upper part of the device 210 is positioned closer to the leg L than the lower part of the device), such that the leg L is disposed within the interior region 213 defined by the inner surface 212 and the indexing portion 230 is positioned below the kneecap K. The fitting device 210 can then be slid upwardly along the leg L until the curved edge 231 engages and/or receives the kneecap K. In other embodiments, the fitting device 210 can be positioned such that the kneecap K is received within the curved edge 231 of the indexing portion 230 while the leg L remains spaced from of the interior region 213 defined by the inner surface 212. The fitting device can then be rotated downwardly such that a portion of the leg L is received within the interior region 213 defined by the inner surface 212.

When the fitting device 210 is disposed about the patient's leg L with the patient's kneecap K disposed within the curved edge 231, as shown in FIG. 7, the first electrode 250A is positioned above or adjacent the superficial peroneal nerve SP of the leg L (see, e.g., FIG. 8). Similarly, the second electrode 250B is positioned above or adjacent the deep peroneal nerve DP of the leg L (see, e.g., FIG. 8). In this manner, the fitting device 210 can be used to position the electrodes 250A, 250B at a predetermined location, orientation and/or position on the patient's leg L. Similarly stated, the indexing portion 230 of the fitting device 210 is positioned and/or oriented relative to the attachment portion 220 of the fitting device 210 such that when the fitting device 210 is disposed about the patient's leg L with the patient's kneecap K disposed within the curved edge 231, the electrodes 250A, 250B are disposed adjacent the leg L at a predetermined position and/or orientation relative to the kneecap K.

As shown by the arrow BB in FIG. 5, after the fitting device 210 is positioned as desired about the patient's leg L, the actuator 240B is moved from a first position (see, e.g., FIG. 4) to a second position (see, e.g., FIG. 5). The actuator 240A can be similarly moved, however, for clarity only the movement of actuator 240B is discussed herein. When the actuator 240B is in the first position, the distal end portion 244B of the actuator 240B is disposed adjacent the electrode 250B such that the electrode 250B remains coupled to the coupler 222. In some embodiments, when the actuator 240B is in the first position, the distal end portion 244B of the actuator 240B is disposed within the interior region 213 defined by the inner surface 212. In other embodiments, when the actuator 240B is in the first position, the distal end portion 244B of the actuator 240B is disposed within the lumen 216.

When the actuator 240B is in the second position, the distal end portion 244B of the actuator 240B is disposed within the interior region 213 defined by the inner surface 212 and is in contact with the electrode 250B such that the electrode 250B is spaced apart from the coupler 222. Said another way, when the actuator 240B moves from its first position to its second position as illustrated by the arrow BB in FIG. 5, the distal end portion 244B of the actuator 240B decouples the electrode 250B from the coupler 222. Moreover, when the actuator 240B moves from its first position to its second position, the distal end portion 244B of the actuator 240B moves the electrode 250B into contact with the leg L. In this manner, the fitting device 210 can position the electrodes 250A, 250B in their desired locations on the patient's leg L. After the electrodes 250A, 250B are positioned on the patient's leg L, the fitting device 210 is removed from the leg L, leaving the electrodes 250A, 250B in position on the leg L, as shown in FIG. 8.

As described above, the fitting device 210 can be constructed from any suitable material or combination of materials. For example, in some embodiments, at least a portion of the attachment portion 220, at least a portion of the indexing portion 230, and/or at least a portion of the fitting device 210 disposed therebetween can be constructed from a rigid material, a flexible material or a semi-flexible material. Similarly stated, in some embodiments, at least a portion of the attachment portion 220, at least a portion of the indexing portion 230 and/or at least a portion of the fitting device 210 disposed therebetween can be constructed from an inelastic material. In this manner, the geometrical relationship between the attachment portion 220 and the indexing portion 230 can be maintained, such that the fitting device 210 can be used to repeatably and/or accurately position the electrodes 250A, 250B at a predetermined position and/or orientation relative to the kneecap K, as described above. Similarly stated, in this manner, the distance between the attachment portion 220 and the indexing portion 230 can be substantially constant or fixed.

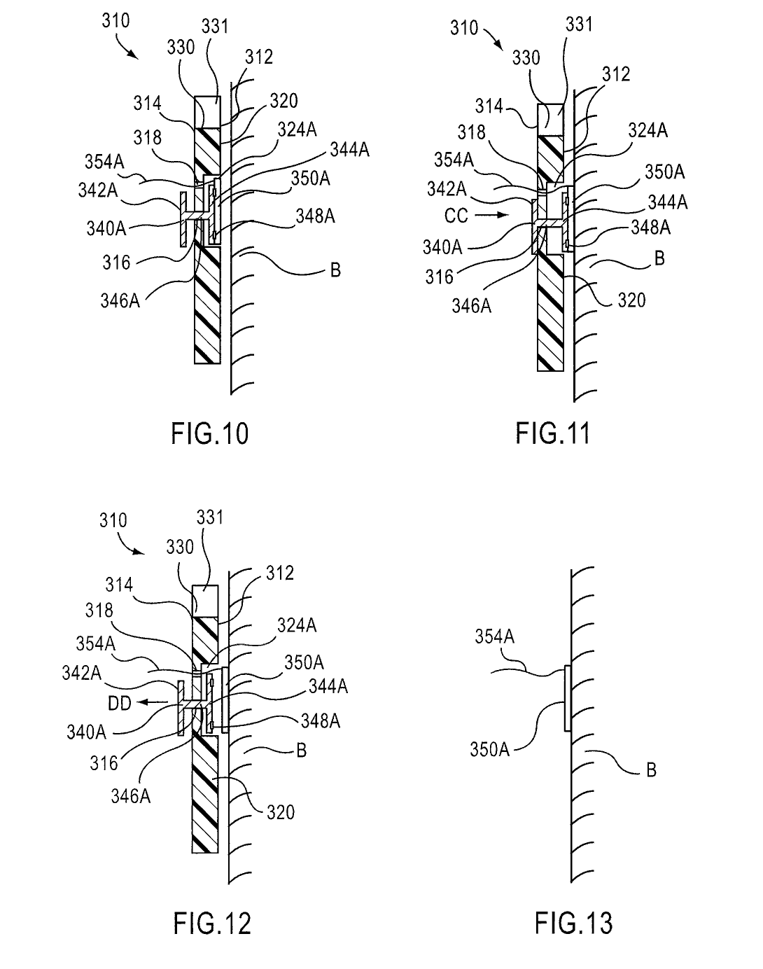

FIGS. 9-13 illustrate a medical device 300 according to an embodiment of the invention. FIG. 9 is a perspective rear view of the medical device 300. FIGS. 10-13 are partial cross-sectional views of the medical device 300 taken along line X-X in FIG. 9 in a first configuration, a second configuration, a third configuration and a fourth configuration, respectively. The medical device 300 includes a fitting device 310, two actuators 340A, 340B (see FIGS. 10-13) and two electrodes 350A, 350B. Only actuator 340A, electrode 350A and their associated structure are illustrated in FIGS. 10-13 for the sake of clarity.

The fitting device 310 has an inner surface 312, an outer surface 314, an attachment portion 320, an indexing portion 330 and two attachment straps 326. The inner surface 312 of the fitting device 310 defines an interior region 313 within which a portion of a patient's body B can be disposed. As described above, in some embodiments, the inner surface 312 of the fitting device 310 can have a shape that corresponds to a shape of the patient's body B (e.g., the patient's hand, leg, foot, arm or the like). In this manner, the fitting device 310 can be disposed about the portion of the body B such that the inner surface 312 is in contact with and/or is adjacent the body B.

When the fitting device 310 is disposed about the portion of the body B, the straps 326 can extend around the portion of the body B to secure the fitting device 310 in position. Each strap 326 includes a coupler 327 configured to removably couple the ends of the straps 326 together. The couplers 327 can be, for example, a hook and loop fastener, a snap fastener, an adjustable buckle fastener or the like. The straps 326 can be constructed from any suitable flexible material, such as, for example, an elastic material or an inelastic material.

As shown in FIG. 9, the attachment portion 320 of the fitting device 310 includes two recesses 324A, 324B defined by the inner surface 312 of the fitting device 310. As shown in FIGS. 10-13, the fitting portion 310 defines a first lumen 316 and a second lumen 318, each of which are in communication with the recess 324A. Although only the structure relating to recess 324A is shown and described herein, in some embodiments, the fitting portion 310 also defines similar lumens in communication with recess 324B. As shown in FIGS. 9 and 10, when the medical device 300 is in its first configuration, the electrode 350A is disposed within recess 324A. As shown in FIG. 10, the electrode 350A is disposed within the recess 324A such that the electrode 350A is in contact with a distal end portion 344A of the actuator 340A, as described in more detail herein. Moreover, the electrode 350A is disposed within the recess 324A such that a lead wire 354A of the electrode 350A extends through the second lumen 318.

As shown in FIG. 9, the size and shape of the recess 324A corresponds to the size and shape of the electrode 350A, and the size and shape of the recess 324B corresponds to the size and shape of the electrode 350B. This arrangement allows the electrodes 350A, 350B to be “loaded” into the fitting device 310 in a predetermined orientation and/or position within the fitting device 310. Said another way, in some embodiments, the electrode 350A can be uniquely associated with the recess 324A and the electrode 350B can be uniquely associated with the recess 324B. Thus, for example, in embodiments in which the electrode 350A has different performance characteristics (such as, for example, the polarity) than the electrode 350B, the recesses 324A and 324B can be sized and/or shaped to prevent the electrodes 350A and 350B from being improperly positioned within the fitting device 310.

The indexing portion 330 of the fitting device 310 defines a curved edge 331 having a shape and/or size that corresponds to a shape of a portion of the patient's body B. For example, in some embodiments, the curved edge 331 can have a shape and/or size corresponding to a kneecap, an ankle, a wrist, an elbow, a thumb or any other suitable portion of the body for indexing the position of the fitting device 310. Accordingly, the indexing portion 330 can receive a portion of the patient's body to position the fitting device 310 about the patient's body B at a predetermined location, orientation and/or position. In this manner, when the fitting device 310 is disposed about the body B with a portion of the patient's body B disposed within the curved edge 331, the electrodes 350A, 350B are positioned at a predetermined location, orientation and/or position on the patient's body B.

As shown in FIGS. 10-13, the actuator 340A has a proximal end portion 342A, a central portion 346A, and a distal end portion 344A. The central portion 346A of the actuator 340A is movably disposed within the first lumen 316 defined by the fitting device 310. The proximal end portion 342A of the actuator 340A is disposed outside of the interior region 313 defined by the inner surface 312. Said another way, the proximal end portion 342A of the actuator 340A is accessible to a user when the fitting device 310 is disposed about the portion of the patient's body B. Accordingly, as described herein, a user can manipulate the proximal end portion 342A of the actuator 340A to move the actuator 340A within the first lumen 316 relative to the fitting device 310 when the fitting device 310 is disposed about the patient's body B.

The distal end portion 344A of the actuator 340A includes a coupler 348A configured to removably couple the electrode 350A to the actuator 340A. The coupler 322 can be, for example, a layer of adhesive, a hook and loop fastener, a snap fastener, a magnetic fastener or any other mechanism suitable for removably coupling the electrode 350A to the actuator 340A. In some embodiments, for example the coupler 322 can be a band of Velcro® configured to engage a cloth backing (not shown in FIGS. 10-13) of the electrode 350A to removably couple the electrode 350A to the actuator 340A.

In use, the fitting device 310 is disposed about a portion of the body B such that a portion of the body B is received within the indexing portion 330 of the fitting device 310, as described above. When the fitting device 310 is positioned as desired about the patient's body B, the actuator 340A is moved from a first position (see, e.g., FIG. 10) to a second position (see, e.g., FIG. 11), as shown by the arrow CC in FIG. 11. The actuator 340B can be similarly moved, however, for clarity only the movement of actuator 340A is discussed herein. When the actuator 340A moves from its first position to its second position the distal end portion 344A of the actuator 340A moves the electrode 350A into contact with the body B. In this manner, the fitting device 310 can position the electrodes 350A, 350B in their desired locations on the patient's body B. In some embodiments, the electrodes 350A, 350B can include a conductive hydrogel (not shown in FIGS. 10-13) to removably couple the electrodes 350A, 350B to the body B.

After the electrodes 350A, 350B are positioned on the patient's leg L, the actuator 340A is moved from the second position back to the first position, as shown by the arrow DD in FIG. 12. When the actuator 340A moves from its second position back to its first position the distal end portion 344A of the actuator 340A is decoupled from the electrode 350A, thus leaving the electrode 350A positioned on the body B. In some embodiments, the retention strength of the conductive hydrogel can be higher than the retention strength of the coupler 348A to ensure that when the actuator 340A moves from its second position back to its first position the electrode 350A is not removed from the body B. In some embodiments, for example, the area and/or the composition of the coupler 348A can be selected to ensure that the electrode 350A is coupled to the distal end portion 344A of the actuator 340A with a relatively weak bond.

As shown in FIG. 13, the fitting device 310 can then be removed from the body B, thereby leaving the electrode 350A in position on the body B. During removal the lead wire 354A of the electrode can be passed through the second lumen 318.

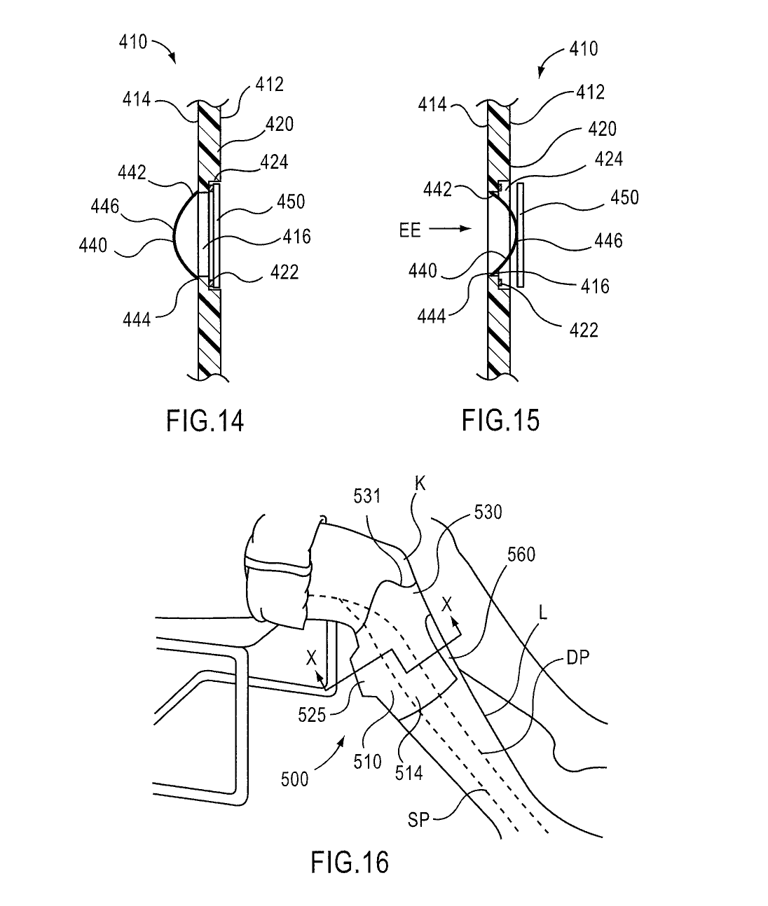

Although the actuators are shown and described above as being actuated by manipulating a proximal end portion of the actuator, in some embodiments, an actuator can be actuated by manipulating any suitable portion of the actuator. For example, FIGS. 14 and 15 show a cross-sectional view of a portion of a fitting device 410 according to an embodiment of the invention in a first configuration and a second configuration, respectively. The fitting device 410 includes an inner surface 412, an outer surface 414, an attachment portion 420, and an actuator 440. As described above, the fitting device 410 can be disposed about the portion of the body (not shown in FIGS. 14 and 15) such that the inner surface 412 is in contact with and/or is adjacent the body. For example, in some embodiments, the inner surface 412 of the fitting device 410 can have a shape that corresponds to a shape of the patient's body.

The attachment portion 420 of the fitting device 410 includes a recess 424, defined by the inner surface 412 of the fitting device 410 and a lumen 416 defined by the fitting device 410 in communication with the recess 424. A coupler 422 is disposed on the inner surface 412 of the fitting device 410 within the recess 424 to removably couple an electrode 450 within the recess 424. As described above, the coupler 422 can be, for example, a layer of adhesive, a hook and loop fastener, a snap fastener, a magnetic fastener or any other mechanism suitable for removably retaining the electrodes 450 within the recess 424.

In some embodiments, the size and shape of the recess 424 corresponds to the size and shape of the electrode 450. This arrangement allows the electrode 450 to be “loaded” into the fitting device 410 in a predetermined orientation and/or position. Similarly, in some embodiments, the recess 424 can be uniquely associated with a particular type of electrode 450.

The actuator 440 has a first end portion 442, a central portion 446, and a second end portion 444. The first end portion 442 and the second end portion 444 are coupled to the outer surface 414 of the fitting device 410. The actuator is movably coupled to the fitting device 410 between a first configuration (FIG. 14 and a second configuration FIG. 15). When the actuator 440 is in the first configuration, the central portion 446 of the actuator 440 is disposed apart from the electrode 450. Said another way, when the actuator 440 is in the first configuration, outer surface 414 of the fitting device 410 is disposed between the central portion 446 of the actuator 440 and the electrode 450.

When the actuator 440 is in the second configuration, the central portion 446 of the actuator is in contact with the electrode 450. In this manner, when the actuator 440 moves from its first configuration to its second configuration, as illustrated by the arrow EE in FIG. 15, the central portion 446 of the actuator 440 is disposed through the lumen 416 and into contact with the electrode 450. Moreover, the central portion 446 of the actuator 440 decouples the electrode 450 from the coupler 422 and moves the electrode 450 into contact with the portion of the body (not shown in FIGS. 14 and 15). In this manner, the fitting device 410 can position the electrode 450 in a desired location on the portion of the body.

In some embodiments, the actuator 440 can be constructed from a resilient material, such as, for example, a thin metallic strip, a polymer or the like. In this manner, when the actuator 440 is in its second configuration and when the user releases the actuator 440, the central portion 446 of the actuator will return to the first configuration as shown in FIG. 14. Said another way, the actuator 440 can be biased in the first configuration.

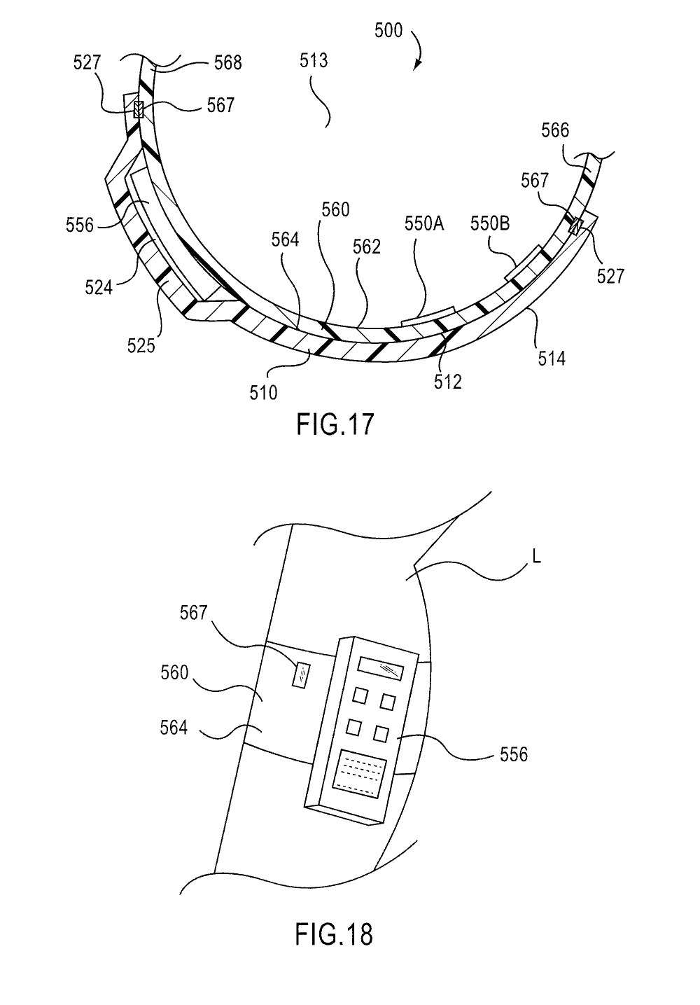

FIGS. 16-18 illustrate a medical device 500 according to an embodiment of the invention. FIG. 16 is a perspective side view of the portion of the medical device 500 positioned on a patient's leg L. FIG. 17 is a cross-sectional view of the medical device 500 taken along line X-X in FIG. 16. FIG. 18 is a perspective rear view of a portion of the medical device 500 positioned on the patient's leg L after the fitting device 510 has been removed. The medical device 500 includes a fitting device 510, a sleeve 560 and an electrical stimulator 556.

As shown in FIG. 17, the sleeve 560 includes a first end portion 566, a second end portion 568, an inner surface 562 and an outer surface 564. The sleeve 560 can be disposed about the leg L such that the inner surface 562 of the sleeve is in contact with the leg L. The sleeve 560 can be removably coupled to the leg L by couplers (not shown in FIG. 17) disposed at the first end portion 566 of the sleeve 560 and the second end portion 568 of the sleeve 560. The couplers can include, for example, a hook and loop fasteners, a snap fastener, an adjustable buckle fastener or the like. In some embodiments, for example the couplers can be mating Velcro® strips.

Two electrodes 550A, 550B of the type shown and described above are fixedly coupled to the inner surface 562 of the sleeve 560. In some embodiments, the electrodes 550A, 550B can be positioned on the inner surface 562 of the sleeve 560 such that when the sleeve 560 is disposed about the patient's leg L, the electrode 550A is disposed adjacent the superficial peroneal nerve and the electrode 550B is disposed adjacent the deep peroneal nerve. In other embodiments, the electrodes 550A, 550B can be positioned on the inner surface 562 of the sleeve 560 such that when the sleeve 560 is disposed about the patient's leg L, the electrodes 550A, 550B can be positioned and/or oriented adjacent any anatomical feature of the patient's body (e.g., nerves, muscles, or the like). In some embodiments, the inner surface 562 of the sleeve 560 can include a recess or multiple recesses (not shown in FIG. 17) within which the electrodes 550A, 550B can be disposed. In this manner, the surface of the electrodes 550A, 550B can be flush with the inner surface 562 of the sleeve 560 when the sleeve 560 is disposed about the patient's leg L.

An electronic stimulator 556 is coupled to the outer surface 564 of the sleeve 560 and is electronically coupled to the electrodes 550A, 550B. The electronic stimulator 556 can provide electrical signals to the electrodes 550A, 550B as a part of a therapeutic program. For example, in some embodiments, the electronic stimulator 556 can provide electrical signals to the electrodes 550A, 550B to cause dorsiflexion of the ankle in patients who have lost the ability to dorsiflex following neurological injury. The stimulator 556 can be any stimulator suitable for providing electrical signals to the electrodes 550A, 550B. For example, in some embodiments, the electronic stimulator 556 can be capable of providing a signal of between 0.2 volts and 1.0 volts at a frequency of between 25 pulses per second and 35 pulses per second.

As shown in FIGS. 16 and 17, the fitting device 510 has an inner surface 512, an outer surface 514, an indexing portion 530 and a stimulator housing portion 525. The inner surface 512 of the fitting device 510 defines an interior region 513 within which the sleeve 560 can be disposed. The inner surface 512 of the fitting device 510 includes two couplers 527 configured to be removably coupled to corresponding couplers 567 disposed on the outer surface 564 of the sleeve 560. In this manner, the sleeve 560 can be removably coupled to the fitting device 510. Moreover, the couplers 527 and 567 can be positioned on the fitting device 510 and the sleeve 560, respectively, such that the sleeve 560 can be removably coupled to the fitting device 510 in a predetermined location and/or orientation with respect to the indexing portion 530 of the fitting device 510. In this manner, as described in more detail herein, the fitting device 510 can be used to repeatably position the sleeve 560 about the patient's leg L in a predetermined location and/or orientation.

The couplers 527 and 567 can be any suitable mechanism for removably coupling the sleeve 560 to the fitting device 510. For example, in some embodiments, the couplers 527 and 567 can be hook and loop fasteners. In other embodiments, the couplers 527 and 567 can be snap fasteners. In yet other embodiments, the couplers 527 and 567 can be zippers. Although shown as including two sets of mating couplers 527 and 567, in some embodiments, the fitting device 510 and the sleeve 560 can include a single coupler.

In some embodiments, the inner surface 512 of the fitting device 510 can have a shape that corresponds to a shape of the patient's leg L. In this manner, the fitting device 510 can be disposed about the leg L such that the inner surface 512 is in contact with and/or is adjacent the leg L.

The indexing portion 530 of the fitting device 510 defines a curved edge 531 having a shape that corresponds to a shape of the patient's kneecap K. Accordingly, the indexing portion 530 can receive a portion of the patient's kneecap K to position the fitting device 510 about the patient's leg L at a predetermined location, orientation and/or position. In this manner, when the fitting device 510 is disposed about the patient's leg L with the patient's kneecap K disposed within the curved edge 531, the sleeve 560, and therefore the electrodes 550A, 550B are positioned at a predetermined location, orientation and/or position on the patient's leg L.

The stimulator housing portion 525 defines a recess 524 within which the electronic stimulator 556 can be disposed when the sleeve 560 is coupled to the fitting device 510, as shown in FIG. 17. In some embodiments, the stimulator housing portion 525 can be constructed from a rigid material to provide a protective covering for the electronic stimulator 556 when the sleeve 560 is being disposed about the leg L and/or when the medical device 500 is being stored. In some embodiments, the stimulator housing portion 525 can include an electrical connector (not shown in FIGS. 16 and 17) configured to electronically couple the electronic stimulator 556 to an external device, such as, for example, a battery charger, a communications device or the like. In this manner, the electronic stimulator 556 can be recharged, reprogrammed or the like, when the sleeve 560 is stored within the fitting device 510.

In use, the sleeve 560 is coupled to the fitting device 510 and the fitting device 510 is disposed about the patient's leg L. When the fitting device 210 is disposed about the patient's leg L such that the patient's kneecap K is within the curved edge 531, the sleeve 560 is positioned such that the first electrode 550A is positioned above or adjacent the superficial peroneal nerve SP of the leg L and the second electrode 250B is positioned above or adjacent the deep peroneal nerve DP of the leg L. In this manner, the fitting device 510 can be used to position the sleeve 560 at a predetermined location, orientation and/or position on the patient's leg L. Similarly stated, the indexing portion 530 of the fitting device 510 is positioned and/or oriented relative to the couplers 527 of the fitting device 510 such that when the fitting device 510 and the sleeve 560 are disposed about the patient's leg L with the patient's kneecap K disposed within the curved edge 531, the sleeve 560 is disposed adjacent the leg L at a predetermined position and/or orientation relative to the kneecap K.

After the fitting device 510 is positioned as desired about the patient's leg L, the sleeve 560 is coupled about the patient's leg L. The sleeve 560 can be removably coupled to the leg L by couplers (not shown in FIG. 17) disposed at the first end portion 566 of the sleeve 560 and the second end portion 568 of the sleeve 560, as described above. The fitting device 510 is then decoupled from the sleeve 560 by manipulating the couplers 527 and 567. For example, in some embodiments, the fitting device 510 can be decoupled from the sleeve 560 by unzipping the couplers 527 and 567. The fitting device 510 can then be removed from the patient's leg L, as shown in FIG. 18, leaving the sleeve 560 positioned about the leg L for therapy.

Upon completion of the therapy, the fitting device 510 can be used to remove the sleeve 560 from patient's leg, by following the above operations described above in the reverse order. In this manner, the sleeve 560 can be stored within the fitting device 510 when not being used for therapy.

The sleeve 560 can be constructed from any material or combination of materials suitable for being disposed about the patient's body and for containing the electronic stimulator 556 and/or electrodes 550A, 550B. For example, in some embodiments, the sleeve 560 can be a flexible garment constructed from a polymer and/or natural materials. In some embodiments, the sleeve 560 can include an inelastic portion and an elastic portion. For example, in some embodiments, the portions of the sleeve 560 that include the couplers 567 and the electrodes 550A, 550B can be constructed from an inelastic material and the end portions 566, 568 of the sleeve 560 can be constructed from an elastic material. In this manner, when the sleeve 560 is coupled to the fitting device 510, the position of the electrodes 550A, 550B relative to the indexing portion 530 of the fitting device 510 can be maintained in a predetermined location and/or orientation. Said another way, by constructing a portion of the sleeve 560 from an inelastic material, the sleeve 560 will not stretch when in use, thereby causing the electrodes 550A, 550B to move out of their desired position. Conversely, end portions 566, 568 can be constructed from an elastic material such that the sleeve 560 can be securely coupled about the patient's leg L.

The fitting device 510 can be constructed from any suitable material. For example, in some embodiments, the fitting device 510 can be a constructed from a plastic, such as Nylon. In other embodiments, the fitting device 510 can be constructed from a composite material, such as fiberglass. In yet other embodiments, the fitting device 510 can be constructed from several different materials, such as, for example, rigid plastic, metallic materials, foam padding, cloth, elastic materials or the like.

Although the medical device 500 is shown and described above as including one fitting device 510 and one sleeve 560, in other embodiments, the medical device 500 can include multiple fitting devices 510 and/or sleeves 560. For example, in some embodiments a kit can include a fitting device that includes an indexing portion, similar to those shown and described above, that is custom-fit for a particular patient. The kit can also include multiple sleeves, each having a different configuration of electrodes. For example, one sleeve of the kit can include a single electrode for use in a first therapeutic regiment. A second sleeve of the kit can include multiple electrodes for use in a second therapeutic regiment. In some embodiments, a second sleeve can include electrodes of a different type, electrodes in different location and/or electrodes in a different orientation. In some embodiments, for example, the electronic stimulator 556 can be removably coupled to the sleeve 560 such that it can be used on multiple different sleeve configurations.

Similarly, in some embodiments, a kit can include multiple different fitting devices 510 of the types shown and described above. For example, in some embodiments, a kit can include different fitting devices corresponding to different patient sizes and/or anatomical relationships. In this manner, a practitioner can select a fitting device to best fit the features of a particular patient.

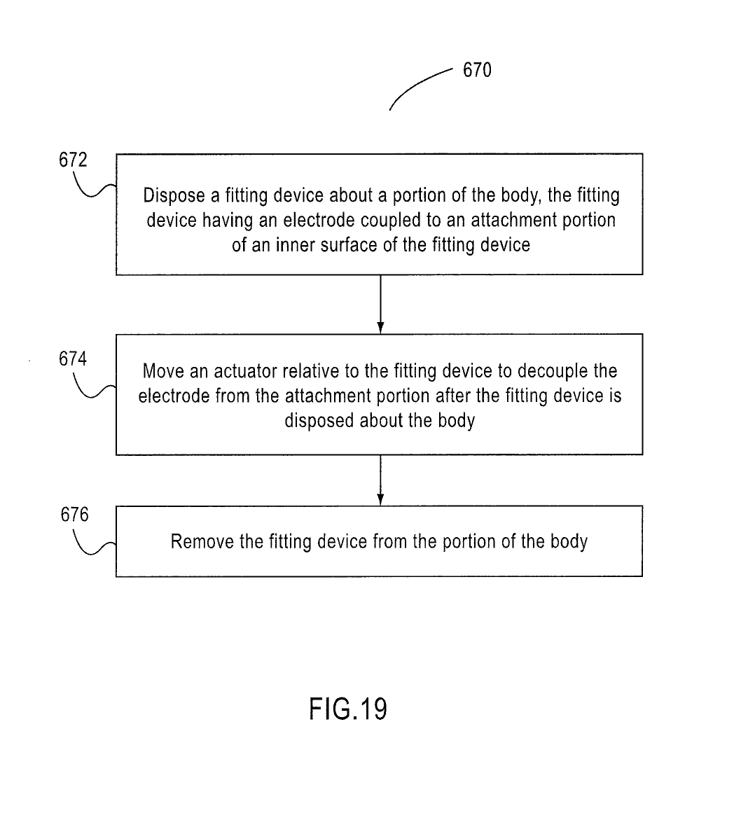

FIG. 19 is a flow chart of a method 670 of placing electrodes on a body according to an embodiment of the invention. The method includes disposing a fitting device about a portion of the body, such as for example, a patient's leg, 672. The fitting device has an electrode coupled to an attachment portion of an inner surface of the fitting device. The fitting device can be any fitting device of the types shown and described above. For example, in some embodiments the fitting device can be fitting device 210 shown and described above with reference to FIGS. 3-8.

After the fitting device is disposed about the portion of the body, an actuator is moved relative to the fitting device to decouple the electrode from the attachment portion of the fitting device, 674. The actuator can be any actuator of the types shown and describe above. For example, in some embodiments, the actuator can be a linear actuator configured to slide within a portion of the fitting device. In other embodiments, the actuator can be configured to rotate relative to the fitting device.

The fitting device is then removed from the body, 676. In this manner, the electrode can be disposed on the patient's body in a desired position and/or orientation without requiring a garment and/or support device to maintain the position and/or orientation of the electrode.

In some embodiments, the moving can include moving the actuator such that the electrode is removably coupled to the portion of the body. In such embodiments, the electrode can include a layer of conductive hydrogel to couple the electrode to the patient's body.

In some embodiments, the moving can include moving the actuator such that a distal end portion of the actuator contacts the electrode to decouple the electrode from the attachment portion of the fitting device. In some embodiments, for example, the moving can include moving the actuator from a first position to a second position. When the actuator is in the first position a proximal end portion of the actuator is spaced apart from an outer surface of the fitting device. When the actuator is in the second position a distal end portion of the actuator contacts the electrode to decouple the electrode from the attachment portion.

In some embodiments, the moving can include moving the actuator such that a distal end portion of the actuator applies a spatially uniform force to an inner surface of the electrode to decouple the electrode from the attachment portion and/or removably coupled to the portion of the body. In some such embodiments, for example, a distal end portion of the actuator can have a shape and/or size corresponding to a shape and/or size of the electrode.

In some embodiments, the disposing can include receiving a second portion of the body within an indexing portion of the fitting device such that the fitting device is positioned about the first portion of the body at a predetermined location relative to the second portion of the body. For example in some embodiments, the disposing can include receiving a kneecap within an indexing portion of the fitting device such that the fitting device can be positioned at a predetermined location on the patient's leg.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the actuators are shown and described above as being manually and/or mechanically actuatable, in some embodiments, an actuator can be automatically actuated. In some embodiments, for example, an actuator can be an electronic actuator that is automatically actuated to position an electrode on the body when a portion of the body is received within an indexing portion of a fitting device. For example, in some such embodiments, the indexing portion of the fitting device can include a sensor configured to output a signal when the portion of the body is satisfactorily received within the indexing portion. In this manner, the fitting device can be configured to prevent premature and/or erroneous placement of electrodes.

Similarly, in some embodiments, an actuator can be a pneumatic actuator, an electronic actuator and/or a hydraulic actuator. In some embodiments, for example, a medical device can include multiple electrodes and a fitting device can include multiple actuators configured to actuate simultaneously after the fitting device is disposed about the body.

In some embodiments, an actuator can be biased in a certain position. For example, in some embodiments, a fitting device can include a spring or other suitable biasing member such that the actuator is maintained in its first (or “loaded”) position. In other embodiments, a fitting device can include a spring or other suitable biasing member such that the actuator is maintained in its second (or “actuated”) position.

Although the fitting devices 200, 300 and 500 are shown and described above as including an indexing portion, in some embodiments a fitting device can include multiple indexing portions. For example, in some embodiments, a fitting device can include a first indexing portion configured to receive a first portion of a patient's body and second indexing portion configured to receive a second portion of a patient's body, different than the first portion. For example, in some embodiments, a fitting device can include a first indexing portion configured to receive a kneecap and a second fitting portion configured to receive an ankle. In other embodiments, a fitting device can include a first indexing portion configured to receive an elbow and a second fitting portion configured to receive a thumb.

Similarly, although the fitting devices 200, 300 and 500 are shown and described above as being disposed on a patient's leg and having an indexing portion configured to receive a portion of a kneecap, in other embodiments, a fitting device can be configured to be disposed about any portion of a patient's body, such as, for example, the arm, hand, foot, torso, head or the like. Similarly, in some embodiments, the indexing portion can be configured to index the location and/or orientation of the fitting device with respect to any suitable anatomical feature of the body. For example, in some embodiments, an indexing portion can be configured to index the location and/or orientation of the fitting device with respect to a thumb, finger, nose, ankle, wrist, elbow, hand, foot or the like.

Although the fitting devices 200, 300 and 500 are shown and described above as including an indexing portion configured to receive a portion of the body, in some embodiments a fitting device can include any suitable indexing portion. For example, in some embodiment, an indexing portion can include a protrusion configured to be disposed within and/or between a portion or portions of the body. For example, in some embodiments, an indexing portion can include a protrusion configured to be received between the patient's fingers.

Although the fitting devices are shown and described above as being configured to place one or more electrodes on a patient's body, in some embodiments a fitting device can be configured to remove electrodes from the patient's body. For example, in some embodiments a fitting device can include a first actuator for placing an electrode on the patient's body prior to a therapeutic session and a second actuator for removing the electrode from the patient's body after the therapeutic session.

Although the medical devices are shown and described above as including one or more electrodes, in some embodiments, a medical device can include a simulated fitting device that is devoid of actual electrodes. In some embodiments, a simulated fitting device can correspond to an actual fitting device, of the types shown and described above, and can be used, for example, to train a user in the placement of electrodes on and/or the removal of electrodes from a patient's body.

What is claimed is:

1. An apparatus, comprising:
a fitting device configured to be disposed about a portion of a body, an inner surface of the fitting device having an attachment portion configured to removably couple an electrode to the fitting device; and
an actuator coupled to the fitting device, the actuator configured to decouple the electrode from the attachment portion when the fitting device is disposed about the portion of the body.

2. The apparatus of claim 1, wherein at least a portion of the fitting device is a flexible garment.

3. The apparatus of claim 1, wherein:
the portion of the body is a first portion;
the fitting device includes an indexing portion configured to receive a second portion of the body to position the fitting device about the first portion of the body at a predetermined location relative to the second portion of the body.

4. The apparatus of claim 1, wherein the fitting device includes a rigid indexing portion configured to temporarily receive a bony portion of the body to position the fitting device about the portion of the body at a predetermined location relative to the bony portion of the body.

5. The apparatus of claim 1, wherein the attachment portion includes a rigid portion configured to maintain a location of the electrode relative to the portion of the body when the fitting device is disposed about the portion of the body.

6. The apparatus of claim 1, wherein the attachment portion includes a recess defined by the inner surface of the fitting device, the recess configured to receive the electrode such that the electrode is maintained in a predetermined orientation relative to the fitting device.

7. The apparatus of claim 1, wherein the attachment portion includes a recess defined by the inner surface of the fitting device, the recess configured to receive the electrode, the recess having a shape corresponding to a shape of the electrode.

8. The apparatus of claim 1, wherein the attachment portion includes at least one of an adhesive, a hook and loop fastener, a snap fastener or a magnetic fastener.

9. The apparatus of claim 1, wherein the actuator includes a proximal end portion and a distal end portion, the proximal end portion disposed outside of an interior region defined by the inner surface of the fitting device, the distal end portion configured to contact the electrode to decouple the electrode from the attachment portion when the fitting device is disposed about the portion of the body.

10. The apparatus of claim 1, wherein the actuator includes a distal end portion, the distal end portion being spaced apart from the electrode when the actuator is in a first position relative to the fitting device, the distal end portion being in contact with the electrode to decouple the electrode from the attachment portion when the actuator is in a second position relative to the fitting device.

11. The apparatus of claim 1, wherein the actuator includes a distal end portion configured to contact the electrode to decouple the electrode from the attachment portion, the distal end portion of the actuator having a shape that corresponds to a shape of the electrode.

12. The apparatus of claim 1, wherein the actuator is movably coupled to the fitting device.

13. The apparatus of claim 1, further comprising the electrode.

14. An apparatus, comprising:
a fitting device configured to be disposed about a first portion of a body, the fitting device having an indexing portion and an electrode attachment portion,
the indexing portion configured to receive a second portion of the body to position the fitting device about the first portion of the body at a predetermined location,
the electrode attachment portion configured to removably couple an electrode to an inner surface of the fitting device such that the electrode is disposed at a predetermined location relative to the second portion of the body when the fitting device is disposed about the first portion of the body.

15. The apparatus of claim 14, wherein the indexing portion has a shape corresponding to a shape of the second portion of the body.

16. The apparatus of claim 14, wherein the indexing portion is configured to receive at least one of a kneecap, an ankle, an elbow, a thumb, a hip or a shoulder.

17. The apparatus of claim 14, wherein:
at least a portion of the indexing portion is rigid; and
at least a portion of the electrode attachment portion is rigid.

18. The apparatus of claim 14, wherein a distance between the indexing portion and the electrode attachment portion is substantially constant.

19. The apparatus of claim 14, wherein the electrode attachment portion includes at least one of an adhesive, a hook and loop fastener, a snap fastener or a magnetic fastener.

20. The apparatus of claim 14, wherein the electrode is disposed adjacent at least one of the deep peroneal nerve or the superficial peroneal nerve when the fitting device is disposed about the first portion of the body such that the second portion of the body is received within the indexing portion of the fitting device.

21. The apparatus of claim 14, wherein the electrode attachment portion includes a recess defined by the inner surface of the fitting device, the recess configured to receive the electrode, the recess having a shape corresponding to a shape of the electrode.

22. The apparatus of claim 14, further comprising:
an actuator movably coupled to the fitting device, the actuator configured to decouple the electrode from the inner surface of the fitting device when the fitting device is disposed about the first portion of the body.

23. The apparatus of claim 14, further comprising:
an actuator movably coupled to the fitting device and having a proximal end portion and a distal end portion, the proximal end portion disposed outside of an interior region defined by the inner surface of the fitting device, the distal end portion configured to contact the electrode to decouple the electrode from the electrode attachment portion when the fitting device is disposed about the first portion of the body.

24. An apparatus, comprising:
a fitting device configured to be disposed about a portion of a body; and
an actuator coupled to the fitting device, a distal end portion of the actuator including a connector configured to removably couple an electrode to the distal end portion of the actuator, the actuator configured to move the electrode into contact with the portion of the body when the fitting device is disposed about the portion of the body.

25. The apparatus of claim 24, wherein at least a portion of the fitting device is a flexible garment.

26. The apparatus of claim 24, wherein:
the portion of the body is a first portion;
the fitting device includes an indexing portion configured to receive a second portion of the body to position the fitting device about the first portion of the body at a predetermined location relative to the second portion of the body.

27. The apparatus of claim 24, wherein an inner surface of the fitting device defines a recess configured to receive the distal end portion of the actuator and the electrode.

28. The apparatus of claim 24, wherein an inner surface of the fitting device defines a recess configured to receive the distal end portion of the actuator and the electrode, the recess having a shape corresponding to a shape of the electrode.

29. The apparatus of claim 24, wherein:
the fitting device defines a lumen therethrough;
an inner surface of the fitting device defines a recess in communication with the lumen; and
a central portion of the actuator is slidably disposed within the lumen such that the distal end portion of the actuator is within the recess and a proximal end portion of the actuator is disposed outside of an interior region defined by the inner surface of the fitting device.

30. The apparatus of claim 24, wherein the connector includes at least one of an adhesive, a hook and loop fastener, a snap fastener or a magnetic fastener.

31. The apparatus of claim 24, wherein the actuator is movably coupled to the fitting device.

32. The apparatus of claim 24, further comprising the electrode.

33. A method, comprising:
disposing a fitting device about a portion of a body, the fitting device including an electrode coupled to an attachment portion of an inner surface of the fitting device;
actuating an actuator relative to the fitting device to decouple the electrode from the attachment portion after the disposing; and
removing the fitting device from the portion of the body.

34. The method of claim 33, wherein the actuating includes moving the actuator such that the electrode is removably coupled to the portion of the body.

35. The method of claim 33, wherein the actuating includes moving the actuator such that a distal end portion of the actuator contacts the electrode to decouple the electrode from the attachment portion.

36. The method of claim 33, wherein the actuating includes moving the actuator from a first position to a second position, in the first position a proximal end portion of the actuator is spaced apart from an outer surface of the fitting device, in the second position a distal end portion of the actuator contacts the electrode to decouple the electrode from the attachment portion.

37. The method of claim 33, wherein the actuating includes moving the actuator such that a distal end portion of the actuator applies a spatially uniform force to an inner surface of the electrode to decouple the electrode from the attachment portion.

38. The method of claim 33, wherein:
the actuating includes moving the actuator such that the electrode is removably coupled to the portion of the body; and
the removing includes removing the fitting device such that the electrode remains coupled to the portion of the body.

39. The method of claim 33, wherein:
the portion of the body is a first portion; and
the disposing includes receiving a second portion of the body within an indexing portion of the fitting device such that the fitting device is positioned about the first portion of the body at a predetermined location relative to the second portion of the body.

40. The method of claim 33, wherein:
the portion of the body is a portion of a leg; and
the disposing includes receiving a kneecap within an indexing portion of the fitting device such that the fitting device is positioned about the portion of the leg in a predetermined location relative to the kneecap.

41. An apparatus, comprising:
a sleeve having an inner surface and an outer surface, the sleeve configured to be disposed about a first portion of a body;
an electrode coupled to the inner surface of the sleeve; and
a fitting device having an indexing portion and an attachment portion, the indexing portion configured to be disposed about a second portion of the body, the attachment portion configured to removably retain the sleeve within an interior portion of the fitting device such that the electrode is disposed at a predetermined location relative to the second portion of the body when the fitting device is disposed about the first portion of the body.

42. The apparatus of claim 41, wherein a first end portion of sleeve is configured to be removably coupled to a second end portion of the sleeve when the fitting device is disposed about the first portion of the body.

43. The apparatus of claim 41, wherein the inner surface of the sleeve defines a recess configured to receive the electrode such that the electrode is maintained in a predetermined location relative to the indexing portion of the fitting device when the sleeve is retained within the interior portion of the fitting device.

44. The apparatus of claim 41, wherein the inner surface of the sleeve defines a recess configured to receive the electrode, the recess having a shape corresponding to a shape of the electrode.

45. The apparatus of claim 41, further comprising:
a stimulator coupled to the outer surface of the sleeve, the stimulator electronically coupled to the electrode.

46. The apparatus of claim 41, wherein:
the first portion of the body is a portion of a leg; and
the second portion of the body is a kneecap.

47. The apparatus of claim 41, wherein the indexing portion of the fitting device has a shape corresponding to a shape of the second portion of the body.

48. The apparatus of claim 41, wherein at least a portion of the indexing portion of the fitting device is rigid.

49. The apparatus of claim 41, wherein the attachment portion of the fitting device includes at least one of a zipper, a hook and loop fastener, a snap fastener or a magnetic fastener.

50. The apparatus of claim 41, wherein the electrode is disposed adjacent at least one of the deep peroneal nerve or the superficial peroneal nerve when the fitting device is disposed about the first portion of the body.

51. The apparatus of claim 41, wherein:
the electrode is one of a plurality of electrodes, each electrode from the plurality of electrodes coupled to the inner surface of the sleeve such that each electrode from the plurality of electrodes is disposed at a predetermined location relative to the second portion of the body when the fitting device is disposed about the first portion of the body.
